(19) United States
(12) Patent Application Publication
(10) Pub. No.: US 2021/0259621 A1
(43) Pub. Date: Aug. 26, 2021

(54) WEARABLE SYSTEM FOR BRAIN HEALTH MONITORING AND SEIZURE DETECTION AND PREDICTION

(71) Applicant: CORTEXXUS INC., Berkeley, CA (US)

(72) Inventors: David ALVES, Berkeley, CA (US); Babak RAZAVI, Berkeley, CA (US); Ana Margarida DE JESUS ALVES, Berkeley, CA (US)

(73) Assignee: CORTEXXUS INC., Berkeley, CA (US)

(21) Appl. No.: 17/255,549

(22) PCT Filed: Jun. 27, 2019
(86) PCT No.: PCT/US2019/039547

§ 371 Date: Dec. 23, 2020

Related U.S. Application Data
(60) Provisional application No. 62/690,520, filed on Jun. 27, 2018.
(60) Provisional application No. 62/800,194, filed on Feb. 1, 2019.

(51) Int. Cl. A61B 5/00; G10L 25/66; G06K 9/00; A61B 5/369; A61B 7/00; A61B 5/11; G16H 40/67; G16H 50/20; G16H 50/70; G06N 3/08

(52) U.S. Cl. CPC A61B 5/4094; A61B 2562/0219; G06K 9/00255; G06K 9/00335; G06K 9/00536; A61B 5/6803; A61B 5/369; A61B 5/7267; A61B 5/7275; A61B 5/0077; A61B 7/00; A61B 5/746; A61B 5/1126; G16H 40/67; G16H 50/20; G16H 50/70; G06N 3/08; G10L 25/66

(57) ABSTRACT

The present disclosure provides for monitoring brain health and predicting and detecting seizures via a wearable head ap paratus. An exemplary system includes a wearable head apparatus with a plurality of sensors. The system includes a memory device with instructions for performing a method. The method provides for first receiving electroencephalography (EEG) data and/or other data types output by the plurality of sensors. The EEG data includes electrical signals representing brain activity of a user. The method provides for processing the EEG data and/or other data types using a machine learning model to identify a time window of a subset of the EEG data and/or other data types, which represents a seizure. The method provides for tagging the time window as seizure data. A representation of the time window of the EEG data and/or other data types is then output.

### CROSS-REFERENCE TO RELATED APPLICATIONS

This application claims priority under 35 U.S.C. § 119 to and the benefit of U.S. Provisional Patent Application No. 62/800,194, filed Feb. 1, 2019, entitled, “Wearable Seizure Prevention System,”, and of U.S. Provisional Patent Application No. 62/690,520, filed Jun. 27, 2018, entitled, “Wearable System for Brain Health Monitoring and Seizure Detection and Prediction”, the contents of both of which are incorporated herein by reference in their entireties.

### FIELD

The present invention relates to methods and devices for monitoring, detecting, and predicting seizures, and general brain health monitoring.

### BACKGROUND

Seizures, medically termed epileptic seizures, are brief episodes due to abnormal neuronal activity in a person's brain. Neurologically speaking, seizures occur when a group of neurons begin firing in an abnormal, excessive, and synchronized manner. The abnormally synchronous neuron firing causes seizure signs and symptoms which can range from lengthy uncontrolled jerking movement and loss of consciousness to subtle momentary loss of awareness. Approximately 5-10% of people will experience an epileptic seizure during their lifetime, and about half of those people will experience a second seizure. Epilepsy is a diagnosis of recurrent epileptic seizures.

Seizures can occur for many different reasons, due to genetic causes, stress, brain trauma, dehydration, overheating, and drug use, among other reasons. Depending on where the individual is when the seizure occurs, the seizure can expose the individual to various dangers. Besides the seizure itself where the individual is not in full control of their body, seizures are typically followed by a period of disorientation, which can last minutes to hours. Repeated seizures can cause brain atrophy, neuronal loss, and severe neurological damage. Therefore, it is imperative to get medical treatment and help quickly.

Seizures can be both convulsive and non-convulsive. Convulsive seizures occur when the body muscles contract and relax rapidly and repeatedly to cause jerky movement. Non-convulsive seizures do not affect the muscular system and are often characterized by a loss and return to consciousness, or confusion. People affected by non-convulsive seizures, can have non-convulsive seizures multiple times a day. In extreme circumstances, the individual might have non-convulsive seizures hundreds of times each day, which results in extreme disorientation and impaired cognitive function.

Therefore, monitoring, detection, and prediction of seizure activity in those afflicted by epileptic seizures is extremely important. Monitoring, detecting, and predicting seizures can help prevent seizures, prevent potential bodily injuries and even death (called Sudden Unexpected Death in Epilepsy, or SUDEP), identify seizures, and identify the effectiveness of medication, among other reasons. It can be imperative for treatment to monitor a person's epileptic seizure events over an extended period of time, especially while the person is out of the doctor's presence.

However, current devices for detecting and predicting seizures have a number of downsides that do not make it practical for individuals to use them at home or outside of a doctor's presence. Current devices can be extremely expensive and large, making them impractical or impossible for consumer use. Additionally, current devices might be uncomfortable, or have limited functionality and ability to sense when a seizure has occurred or what type of seizure occurred.

### SUMMARY

The present disclosure provides systems and methods for monitoring brain health and brain function. The present disclosure can provide for a brain health system. The system can also be referred to as a brain health monitoring system. An exemplary brain health system, according to an embodiment of the present disclosure, can include a wearable head apparatus, a plurality of sensors, a memory device, and a control system. The memory device can contain machine-readable medium comprising machine executable code. The code can have stored on the machine instructions for performing a method of determining electrical signals of a user of the wearable head apparatus. The control system can be coupled to the memory device and comprise one or more processors. The control system can be configured to execute the machine executable code, which can cause the one or more processors to complete a series of steps. First the processors receive electroencephalography (EEG) data output by the plurality of sensors. The EEG data includes electrical signals representing brain activity of a user. Then, the processors process the EEG data using a machine learning model to identify a time window of a subset of the EEG data representing a seizure or other brain electrical activity of interest.

In some examples, the processors tag the time window of the subset of the EEG data as seizure data or any other activity of interest. Lastly, the processors output a representation of the time window of the EEG data. In some examples, the output representation includes at least one of: an indication that the user is having a seizure, or a prediction that the user will have a seizure.

In some examples, the received data output by the plurality of sensors includes heart rate data, pulse oximetry data, accelerometer data, and any combination thereof.

In some examples, the wearable head apparatus can comprise a pattern. The control system can be further configured to execute the machine executable code to cause the one or more processors to identify a seizure of the user based on at least analysis of the pattern in the data output by the plurality of sensors.

In some examples, the electrical signals can be determined with respect to indications of synchronous neuronal activity (such as those caused by a seizure) in a brain of the user.

In some examples, the machine learning model can be a convolutional neural network.

In some examples, the machine learning model can be trained with labeled data that classifies whether a subject is experiencing a seizure during a subset of the labeled data.

In some examples, the control system can be further configured to execute the machine executable code to cause the one or more processors to input data output from the plurality of sensors attached to the wearable head apparatus to determine the biological signals.

In some examples, the seizure can be convulsive or non-convulsive.

In some examples, the sensors can be electrodes.

In some examples, the wearable head apparatus can be an eyeglass device. The eyeglass device can comprise a frame and a detachable band. A subset or the entirety of the plurality of sensors can be located on the detachable band. Alternatively, or in addition, the eyeglass device can comprise a frame and a pair of detachable earpieces. A subset or the entirety of the plurality of sensors can be located on the pair of detachable earpieces.

In some examples, each sensor of the plurality of sensors is coupled to the wearable head apparatus.

In some examples, the wearable head apparatus further includes a camera configured to record visual data of the user's face. For example, the control system receives visual data output from the camera and processes the visual data using a machine learning model to identify a time window of a subset of the visual data representing a seizure.

In some examples, the control system further determines whether the identified time window of a subset of the visual data corresponds to the identified time window of a subset of the EEG data. The control system further outputs a notification comprising the determination of whether the identified time window of a subset of the visual data corresponds to the identified time window of a subset of the EEG data.

In some examples, the wearable head apparatus further includes a microphone configured to record audio data of the user. For example, the control system receives audio data output from the microphone and processes the audio data using a machine learning model to identify a time window of a subset of the audio data representing a seizure.

In some examples, the control system further determines whether the identified time window of a subset of the audio data corresponds to the identified time window of a subset of the EEG data. The control system further outputs a notification comprising the determination of whether the identified time window of a subset of the audio data corresponds to the identified time window of a subset of the EEG data.

In some examples, the wearable head apparatus further includes an accelerometer configured to record movement data of the user. For example, the control system receives movement data output from the accelerometer and processes the movement data using a machine learning model to identify a time window of a subset of the movement data representing a seizure.

In some examples, the control system further determines whether the identified time window of a subset of the movement data corresponds to the identified time window of a subset of the EEG data. The control system further outputs a notification comprising the determination of whether the identified time window of a subset of the movement data corresponds to the identified time window of a subset of the EEG data.

In another embodiment, the present disclosure provides a method for training data related to brain health and brain activity. The method can comprise receiving the EEG data output by a plurality of sensors attached to a wearable head apparatus. The wearable head apparatus can be worn by a user. The EEG data can be stored in a memory device. The method can then process the EEG data using a machine learning model to identify a time window of a subset of the EEG data representing a seizure. The method can then proceed by tagging the time window of the subset of the EEG data as seizure data. The method can then output a representation of the time window of the subset of the EEG data. The representation can include a tag of the time window as seizure data. The method can then train the machine learning model based on the subset of the EEG data and the tag as seizure data.

In some examples, the method can further comprise receiving a notification from the user that a seizure has occurred during an untagged subset of the EEG data. The untagged subset was not identified as a time window by the machine learning model. The method can then identify the untagged subset, based on the notification from the user, as a time window representing a seizure. The method can then tag the untagged subset as seizure data. The method can then retrain the machine learning model based on the notification.

In other examples, the method can further comprise receiving a notification from the user that a seizure has not occurred during an incorrect time window. The incorrect time window was identified by the machine learning model as representing a seizure. The method can remove, based on the notification from the user, the tag of the incorrect time window as seizure data. The method can retrain the machine learning model based on the notification.

In other examples, the user can have a caretaker. The caretaker can be a nurse, physician, doctor, or personal caregiver of the user.

In other examples, the method can further comprise receiving a notification from the caretaker of the user that a seizure has occurred during an untagged subset of the EEG data.

The untagged subset was not identified as a time window by the machine learning model. The method can then identify the untagged subset, based on the notification from the user, as a time window representing a seizure. The method can then tag the untagged subset as seizure data. The method can then retrain the machine learning model based on the notification.

In other examples, the method can further comprise receiving a notification from the caretaker of the user that a seizure has not occurred during an incorrect time window. The incorrect time window was identified by the machine learning model as representing a seizure. The method can remove, based on the notification from the user, the tag of the incorrect time window as seizure data. The method can train the machine learning model based on the notification.

In other examples, sending an alert to the user can further comprise sending a notification to a mobile device of the user and/or to a mobile device of the caretaker of the user.

Therefore, the present disclosure provides a seizure detection, prediction and monitoring device which can monitor an individual in the individual's daily life and outside of a physician's control. The device can detect both convulsive and non-convulsive seizures. It can discreetly and continuously monitor the individual. The device can detect and predict seizures. An exemplary device can even notify caregivers and emergency services when the device detects or predicts a seizure. A device like this helps patients feel safe and protected, while continuously monitoring patient brain health.

### BRIEF DESCRIPTION OF DRAWINGS

The patent or application file contains at least one drawing executed in color. Copies of this patent or patent application publication with color drawing(s) will be provided by the Office upon request and payment of the necessary fee. The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

The invention will now be described in relation to the following Figures:

FIG. 1 is a schematic view of an exemplary brain health monitoring system according to an exemplary embodiment of the present disclosure;

FIG. 2 is a diagrammatic view of an example of a wearable head apparatus and brain health monitoring system according to an exemplary embodiment of the present disclosure;

FIG. 3 is a schematic perspective view of a wearable head apparatus, represented as an eyeglass device, according to an exemplary embodiment of the present disclosure;

FIG. 4 is a flow chart illustrating a method for monitoring brain function and health, according to an exemplary embodiment of the present disclosure;

FIG. 5 is a diagrammatic view of a health monitoring system and data exchange, according to an exemplary embodiment of the present disclosure;

FIG. 6 is a diagrammatic view of diagrammatic view of a process 600 for training and selecting a machine learning model serving to detect and predict seizures, according to an exemplary embodiment of the present disclosure; and

FIG. 7 is a diagrammatic view of an exemplary seizure detection and prediction model, according to an exemplary embodiment of the present disclosure.

FIG. 8 shows an exemplary eyeglass device, according to an embodiment of the present disclosure.

FIG. 9 shows an exemplary eyeglass device, according to an embodiment of the present disclosure.

FIG. 10 shows an exemplary eyeglass device, according to an embodiment of the present disclosure.

FIGS. 11-12 show exemplary electrode data, according to an experimental protocol conducted in accordance with the present disclosure.

FIG. 13A shows an exemplary embedded, removable electrode, according to an embodiment of the present disclosure.

FIG. 13B shows an exemplary removable electrode, according to an embodiment of the present disclosure.

FIG. 13C shows an exemplary removable, repositionable electrode, according to an embodiment of the present disclosure.

FIG. 13D shows an exemplary removable, repositionable electrode, according to an embodiment of the present disclosure.

FIG. 13E shows a cutaway view of an exemplary removable, repositionable electrode, according to an embodiment of the present disclosure.

FIG. 13F shows a cutaway view of an exemplary removable, repositionable electrode, according to an embodiment of the present disclosure.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION OF DRAWINGS

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

The present disclosure is directed towards a brain health system that continuously monitors data input from sensors on a wearable head apparatus. The wearable head apparatus can be worn by a person. The sensors constantly send data to a mobile device of the user and a remote server. Data can be sent by Bluetooth, or Wi-Fi, or any other electronic method of transmitting information. The mobile device and the remote server can analyze the data to determine whether the data contains biological data signifying that the user has undergone, is currently undergoing, or is about to undergo a seizure. The brain health system can notify the user accordingly that the user has undergone, is currently undergoing, or is about to undergo a seizure. The brain health system can also notify a caretaker for the user.

In another exemplary embodiment, the present disclosure provides for a machine learning model which can receive data from the brain health monitoring system. The machine learning model can identify whether a set of data identifies a seizure. Continuous updating of the data available to the machine learning model can ensure that the model will grow in accuracy over time. Additionally, the machine learning model can accept input from the user and/or a caretaker of the user. The user and/or caretaker can identify whether the machine learning model correctly identified a seizure, incorrectly identified a seizure, or failed to identify a seizure. Therefore, this additional closed-loop human verification of the events can further and adaptively increase the accuracy of the machine learning model.

Therefore, an exemplary brain health monitoring system, according to an embodiment of the present disclosure, provides for an extremely accessible method of identifying seizures in a user. The system can quickly provide notifications of seizure events to the user and can provide for medical attention to the user. The system provides a small and easily portable wearable head apparatus which can be worn at all times. The system is non-invasive and can work while on Wi-Fi or without connection to Wi-Fi, or any other means of electronic communication. This provides for constant protection of the user during every day activities and outside of a hospital environment.

In addition to the advantages of the exemplary brain monitoring health system, the exemplary machine learning model can train biological data on the user so that the model adapts to the biological indicators of the specific user. Machine learning models benefit greatly from ground-truthing the system to help the system identify whether its classifications are accurate. The present disclosure provides for a simple closed-loop method for the user and caretakers of the user to help increase the accuracy of the system. Thus, the present application provides for a highly, accurate seizure diagnosis system which can be tailored to the biological factors of individual users.

### Brain Health Monitoring System

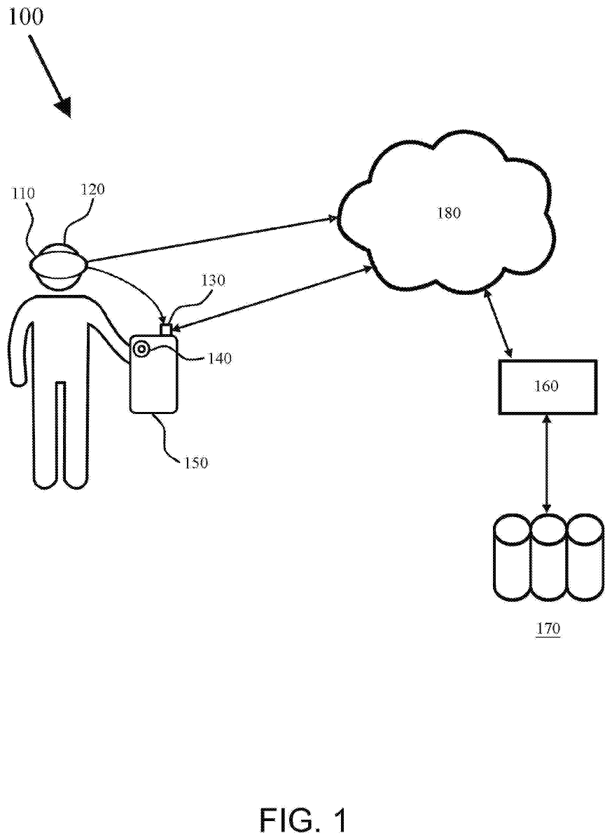

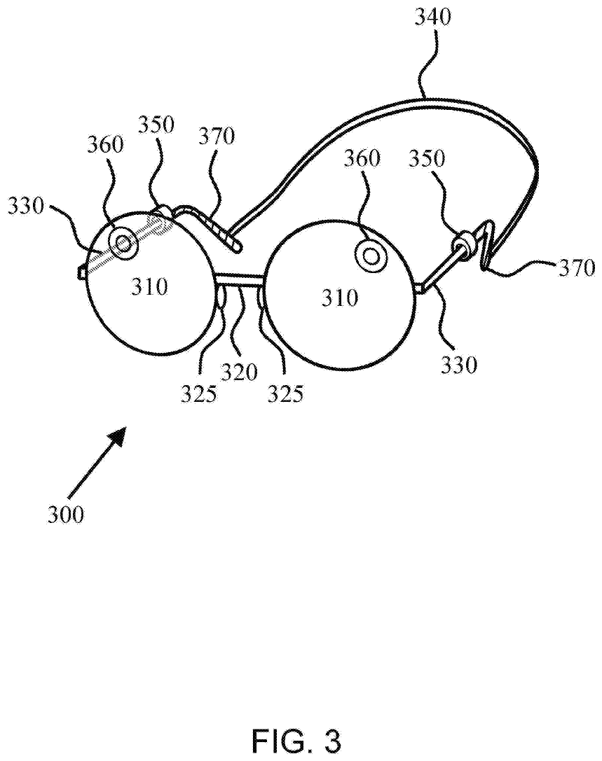

FIG. 1 is a schematic view of an exemplary brain health monitoring system 100 according to an exemplary embodiment of the present disclosure. The brain health monitoring system 100 includes a wearable head apparatus 110; a user 120; a communication link 130; a camera 140; a mobile device 150; a remote server 160; a memory device 170; and a network 180. The wearable head apparatus 110 is a device which sits on the user's 120 head and measures brain activity of the user 120. The wearable head apparatus 110 can have many embodiments, including an eyeglass device, a helmet, a hat, a headband, a facemask, or any other object which attaches to a user's 120 head and measures brain or other biological activity of the user 120 or any activity from the surroundings. The wearable head apparatus 110 is discussed further with regards to FIG. 3. Referring back to FIG. 1, the wearable head apparatus 110 can be configured to communicate with a mobile device 150 through or a network 180 or without a network 180. The mobile device can also be configured to communicate to a remote server 160 through a network 180.

The wearable head apparatus 110 can communicate with the communication link 130 of a mobile device 150. The communication link 130 can also communicate with the network 180. The communication link 130 can communicate with the network 180 and the wearable head apparatus 110 in a variety of ways, including via Bluetooth, Wi-Fi, GSM/UMTS and derivatives, radio waves, and any other electronic mode of communication.

An exemplary mobile device, according to an embodiment of the present disclosure can be a cell phone, a portable phone, a tablet device, a laptop device, or any other similar electronic component.

The mobile device 150 also contains a camera 140. The camera 140 can be configured to monitor movement of the user 120 or the user's 120 surroundings. For example, monitoring the user's 120 surroundings can identify stability of the user 102 based on whether the captured video frame is shaking or stable. The mobile device 150 can be any device configured to connect to a network 180 and to send data over the network 180.

The network 180 can be configured to handle transfers of information between a remote server 160, a mobile device 150, and a wearable had apparatus 110, in any order or combination. The remote server 160 can be configured to process data received from the mobile device 150 or the wearable head apparatus 110, or both. For example, the remote server 160 can run a machine-learning model on the data received. In other instances, the machine-learning model can be run on a mobile device, or wearable head apparatus]. The remote server 160 can communicate with a memory device 170 to store either the received data itself or the results of any analysis performed on the data. The saved data can be used to continually improve the algorithms.

In some instances of the present disclosure, additional components can be included in system 100. For example, external cameras and microphones can be mounted on a wall in the user's location. These cameras and microphones can provide ancillary data.

### Electronic Brain Health System

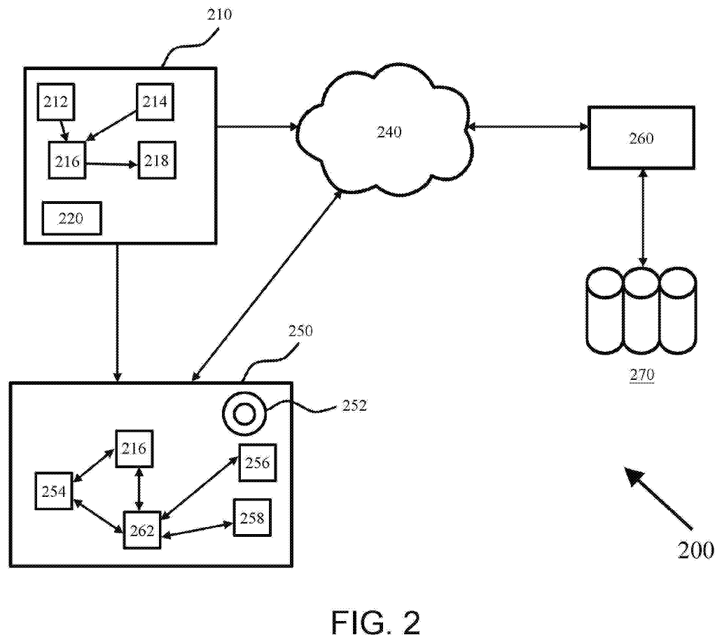

FIG. 2 is a diagrammatic view of an example of an electronic health system 200 according to an exemplary embodiment of the present disclosure. The electronic health system 200 includes a wearable head apparatus 210; a first sensor 212; a second sensor 214; a health application 216; a first signal transmitter 218; a battery 220; a network 240; a mobile device 250; a camera 252; a memory storage 254; a Wi-Fi communication link 256; a Bluetooth communication link 258; a remote server 260; a processor 262; and a remote memory device 270.

The wearable head apparatus 210 contains various components. The first sensor 212 and second sensor 214 operate to collect data from a user. The first sensor and second sensor can be a variety of sensors which can collect biological data. Both the first sensor and the second sensor can be EEG sensors. The biological data from the first sensor 212 and second sensor 214 are sent by the health application 216 to the signal transmitter 218. The health application 216 can be run through a digital signal processor, microcontroller, or other processing component and which operations are controlled by the health application 216. The signal transmitter 218 can operate via Wi-Fi, Bluetooth, radio signals, or any other method of remote communication. The signal transmitter 218 can operate to send the biological data to a remote server 260 through a network 240 or to a mobile device 250.

In a first example, the signal transmitter 218 can send the biological data to the mobile device 250 through the network 240 which can connect to a Wi-Fi communication link 256 on the mobile device 250. In this instance, the signal transmitter 218 can use Wi-Fi to transmit the data. In a second example, the signal transmitter 218 can send the biological data directly to the mobile device 250 by connecting to the Bluetooth communication link 258. In this instance, the signal transmitter 218 can use a Bluetooth connection and Bluetooth signal. There can be one signal transmitter 218 to transmit all types of signals or there can be separate elements for the separate communications.

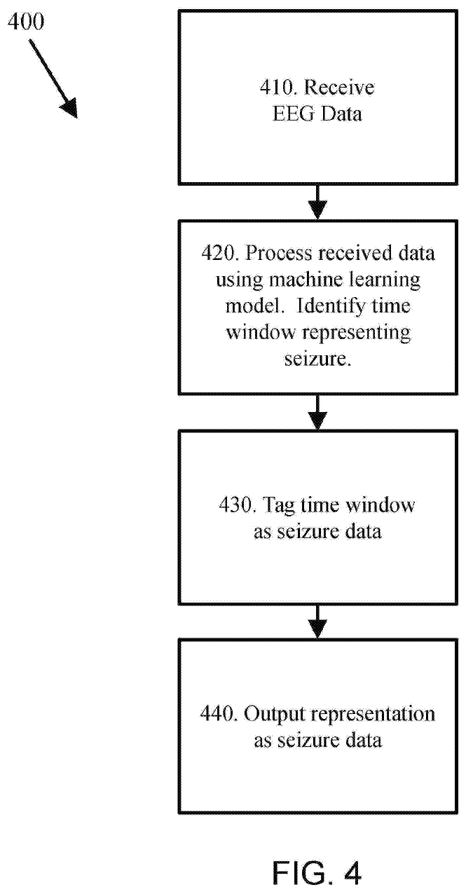

Once the biological data is received by the mobile device 250, the mobile device 250 can operate to process the biological data. The mobile device 250 can have a health application 216 stored on the mobile device 250. This health application 216 can be held in memory storage 254 and run by a processor 262. The processor 262 can be a digital signal processor, microcontroller, or other processing component and which operations are controlled by the health application 216. The health application 216 can run a machine learning model to analyze the biological data. This machine learning model will be discussed later with regards to FIGS. 4-6.

Referring back to FIG. 2, the mobile device 250 can also have a camera 252. In some embodiments of the present disclosure, the camera 252 can be on the wearable head apparatus 210. The camera 252 can collect supplemental data. For example, the camera 252 can collect video or images of the user's environment. The mobile device 250 can also have a microphone configured to collect sound data. The visual and audio data can be held in memory storage 254 and analyzed by the health application 216 to detect whether the user is stable, whether the user has fallen over, whether the user has not moved for a lengthy period of time, whether other people around the user are showing or voicing concern for the user, and conduct other possible analysis. This supplemental data can help determine whether a user has had a seizure at a specific point in time. For example, the user might be convulsing during a seizure and the supplemental data would show a shaky frame. Alternatively, or in addition, the user might be nauseated and unstable directly before or preceding a seizure and the video frame might be unstable. Alternatively, or in addition, the user might experience periods of unconsciousness before, during or after a seizure and the supplemental data might reveal that the user has not moved for a lengthy period of time. Therefore, these examples show that the camera 252 on the mobile device 250 can provide supplemental data to the data collected by the first sensor 212 and the second sensor 214 on the wearable head apparatus 210.

The remote server 260 provides another avenue to process the biological data and the supplemental data. The wearable head apparatus 210 can send the biological data through the network 240 to the remote server 260. Even if the wearable head apparatus 210 sends the biological data to the mobile device 250, the mobile device 250 can transmit the data through the network 240 to the server 260 for processing. The mobile device 250 can also send the supplemental data captured from the camera 252. The remote server 260 can process the data with a machine learning model as will be discussed later with regards to FIGS. 4-6. Referring back to FIG. 2, the remote server can arrange to have the processed data and the original biological data stored on the remote memory storage 270 or in the memory storage device 253 of the mobile device 250.

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known, for example, as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### Data Protocols and Transfer

In some embodiments, a data exchange circuit of the wearable head apparatus and the mobile device can use a wireless protocol, for example: Wi-Fi®, Bluetooth®, GSM or others. In some embodiments, the brain health system may have a unique identifier, to allow the pairing of a mobile device and the wearable head apparatus.

In other embodiments, the wearable head apparatus and the mobile device can utilize wired connections. For example, the data exchange circuit connection to the network is wired. Identification data may be incorporated in the data packets that include the stored signals from the sensors that are sent over the network. The identification can include a serial identity number of the wearable head apparatus. Additionally, biological data obtained during from the user can be time-stamped using data from an internal clock of the mobile device and/or the wearable head apparatus.

In other embodiments, the network comprises at least a wireless local area network (WLAN) and during the step of communication, the wearable head apparatus transmits data to said mobile device via said WLAN. The WLAN may operate according to a communication protocol selected from the Wi-Fi or Bluetooth protocols. A mobile, camera, or other computing device may also be in communication with the local wireless local area network and in the communication step, the wearable head apparatus transmits said data to the mobile device via said wireless LAN.

The LAN may include a server that communicates with at least the wearable head apparatus, and in the communication step, the wearable head apparatus may transmit said data to the mobile device by means of the server. The telecommunication network may further comprise a network of separate remote wireless LANs, the server communicating with at least one server via said remote network, the mobile device also communicating with said server via the remote network.

The information exchanged between the wearable head apparatus, camera, sensor(s), mobile device, and/or the remote server through the interfacing circuits may include data or commands, the data including stored, processed signals from the sensors or raw data from the sensors. Information may be transmitted from the wearable head apparatus to the remote server and, conversely, from the remote server to the wearable head apparatus, as needed. The data can also be a program or software update to store and/or execute by the wearable head apparatus or the mobile device. For example, updates and new firmware may be wirelessly downloaded and installed on the wearable head apparatus.

### Wearable Head Apparatus

FIG. 3 is a schematic perspective view of a wearable head apparatus 300, represented as an eyeglass device 300, according to an exemplary embodiment of the present disclosure. The eyeglass device 300 includes a pair of lenses 310; an eyeglass frame 320; nosepieces 325; eyeglass earpieces 330; detachable band 340; sensors 350; cameras 360; and detachable ear extensions 370.

The lenses 310 can be prescription lenses according to a prescription need of the user. Alternatively, the lenses 310 can provide no eyesight assistance. In some examples, the lenses 310 provide physical protection for the eyes and/or are tinted to filter sunlight. In another example, the lenses 310 can be entirely omitted from the eyeglass device 300, such that the frame 320 connects directly to the eyeglass earpieces 330. In some examples, the frame 320 can be curved around the user's eye so as not to interfere with the user's eyesight.

The eyeglass earpieces 330 and the nosepieces 325 serve to secure the eyeglass device 300 on the user's head. The eyeglass earpieces 330 and the nosepieces 325 can contain sensors 350. In some embodiments, the eyeglass earpieces 330 and the nosepieces 325 can contain embedded sensors 350.

The eyeglass earpieces 330 can also have attached detachable ear extensions 370. The detachable ear extensions 370 can cover part or all of the eyeglass earpieces 330. The detachable ear extensions 370 can be removed from or placed on the eyeglass device 300 by the user. The detachable ear extensions 370 can have embedded or attached sensors 350 to measure additional biological data. A detachable band 340 can also be connected to the eyeglass earpieces 330. The detachable band 340 can be configured to fig snuggly around the user's head. The detachable band 340 can also include sensors 350 configured to measure biological data.

There can be two sensors 350 on the eyeglass device 300 or there can be any number of sensors 350 on the eyeglass device 300 so long as there is at least once sensor 350. The sensors 350 serve to measure biological data of the user. A sensor can also measure one or multiple non-biological data. The same sensor can measure both biological and non-biological data.

Although one exemplary configuration of sensors 350 is shown in FIG. 3, sensors can be located anywhere on the eyeglass device 300. Some embodiments of the present disclosure can also include microphones and additional detachable components. The microphones can be configured to receive audio data of events occurring near a user to monitor the user's interactions. Additional detachable components can include additional sensors or be used for comfort.

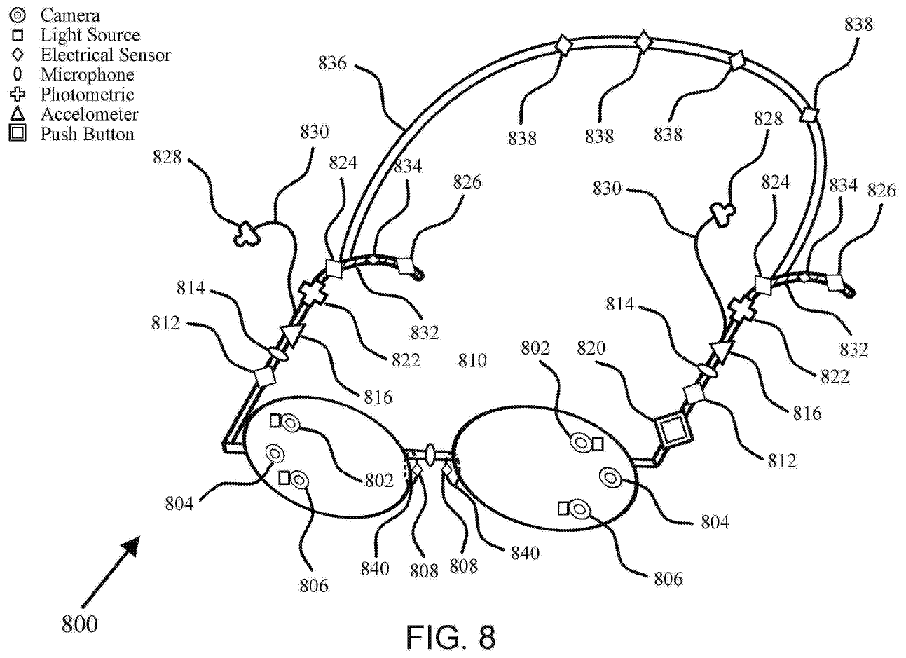

FIG. 8 shows an eyeglass device 800 with all the same features as the eyeglass device 300 shown in FIG. 3. Referring back to FIG. 8, the eyeglass device 800 also includes with possible locations 802-826 for different types of sensors; detachable ear extensions 832 with an electrical sensor 834; detachable band 836 with embedded electrical sensors 838; nose pieces 840; and a wire 830 connecting a mini headphone 828 to the eyeglass device 800.

FIG. 8 shows various locations for sensors. For example, any of the sensors can include a camera, a light source, a light sensor, an electrical sensor, a microphone, a photometric sensor, an accelerometer, and a user input source. Examples follow, detailing what each type of sensor can measure and where each type of sensor can be located. The examples are not meant to be exhaustive and can include variations of locations of sensors on the eyeglass device 800 and types of sensors.

Electrical sensors can include EEG sensors, electromyography (EMG) sensors, electrooculogram (EOG) sensors, electrocardiography (EKG) sensors, and electro-dermal activity (EDA) sensors. These electrical sensors can collect data on the wearer according to their capabilities. Analysis of electrical sensor data can determine normal values and determine how the data differs during a seizure event. This list of electrical sensors is not meant to be exhaustive. The electrical sensors can take any shape or form, be constructed from different materials, coupled with adhesive or conductive material, embedded into the wearable head device independently or in combination with one or more other sensors.

Photometric sensors can include oxygenation sensors, pH sensors, pulse sensors, and/or blood pressure sensors. Analysis of photometric data can determine normal values for this data and can determine how the data differs during a seizure event. This list of photometric sensors is not meant to be exhaustive.

Kinetic sensors can include accelerometers. For example, the accelerometer can detect shaky movement of the wearer and can detect when the wearer is falling. Both shaky movement and a user falling down can indicate that the user is experiencing a seizure event.

Cameras can include video cameras and photographic cameras. These cameras can detect eye movements, blinking, pupil size, skin color, and a heart rate. For example, changes in eye movements, blinking, and pupil size can indicate that a seizure event is occurring. Analysis of camera data can determine normal values and determine how the data differs during a seizure event.

Microphones can detect sound. Increased background noise can indicate that the user is experiencing shaky movements of a seizure event. The microphone can also detect voices from others indicating alarm or concern for a user. In some instances, a microphone can also indicate a warning to the user by sounding an alarm. The microphone can also record biological data such as breathing. Analysis of microphone data can determine normal values and determine how the data differs during a seizure event.

For example, location 802 can include wide-angle cameras pointing towards the eyes and face of a wearer. Wide-angle cameras can detect eye movement, pupil size, blinking, skin color, pulse, facial movements, and facial twitching. Many of these movements can indicate seizures and general wellness of the wearer. Changes in eye movement and pupil size, or eye lids closing, can indicate that a wearer is losing consciousness due to a seizure episode. Pulse (heart rate), for example, can be derived from the wearer's skin color. Location 802 can also include visible or non-visible light sources pointing towards the eyes and face of the wearer. This can help detect visual data from the wearer.

Location 804 can include wide-angle cameras pointing away from the user. This camera can collect data on indirect shaking of the user if the camera view is not stabilized. The camera can collect data indicating that the user has fallen over based on the viewing angle of the camera lens. The camera can also collect data on other people's interactions with the wearer. This collected data can indicate that a wearer has experienced a seizure if, for example, the camera detects that the wearer has fallen over or that other people are approaching the user with concern.

Location 806 can include a wide-angle camera pointing down towards the body of the wearer. For example, the camera can detect skin color, pulse, and shaking or physical movement of the wearer's body and limbs. The camera can also detect a wearer's heart rate through collecting visual data of a pulse beating through the patient's skin. Location 806 can also include visible or non-visible light sources pointing down towards the body of the user. Visible and non-visible light sources can help cameras see in low light situations and collect accurate data.

Location 808 can include electrical sensors on the nose pieces 840. There can be one electrical sensor on each of the nose pieces 840.

Location 810 can include a microphone. The microphone can detect sound from the nasal airflow of the wearer to measure respiration rates. Other respiratory sounds, such as snoring, can also be collected for analysis. The microphone can also collect external sounds including (1) other people talking to wearer asking if the wearer is alright, (2) sounds of the patient falling, (3) sounds of the patient dropping items, and (4) chewing movement.

Location 812, 824 and 826 can include electrical sensors. Location 812 can be located over the temple of the wearer. Location 824 can be over the ear of the wearer. Location 826 can be located behind the ear of the wearer. These locations 812, 824, and 826 can be close to or lay on the skin or hair of the wearer so as to collect accurate electrical sensor data.

Location 814 can be a microphone configured to collect external sounds including (1) other people talking to wearer asking if the wearer is alright, (2) sounds of the patient falling, and (3) sounds of the patient dropping items. This microphone can also be used for a voice recognition alerting and commanding system. For example, if the wearer or caregiver says a keyword, a phone call can be triggered and/or a light source can be flashed. The phone call and/or the light source can alert others of the wearer's situation.

Location 816 can be accelerometers which can be placed over the handles and collect data relating to rotation and translation movement of the wearer. For example, the accelerometer can detect shaky movement of the wearer and can detect when the wearer is falling. Both shaky movement and a user falling down can indicate that the user is experiencing a seizure event.

Location 820 can include a user input source, including, for example, a push button. Location 820 can be on either side of the eyeglass device 800. The push button can also be at other locations such as the rim of an eyeglass embodiment or a detachable component such as the band. The push button can receive input from the wearer. For example, the wearer can press the push button to provide a manual method of alerting. Pushing the push button can trigger a phone call or start flashing lights. Alternatively, or in addition, the push button can mark events of interest for the wearer to later review.

Location 822 can include photometric sensors. These sensors can be on the glasses frame over the wearer's ear and measure oxygenation levels, pH, pulse, and/or blood pressure, or other signals.

The eyeglass device 800 can also include wires 830 connecting to a mini headphone 828. The mini headphone 828 can fit inside the ear of the wearer and transmit warning messages to the wearer. A warning message can indicate that a seizure event is predicted to begin.

The eyeglass device 800 can also include detachable ear extensions 832 with electrical sensors 834. These detachable ear extensions 832 can fit onto the frames of the glasses near location 826. The electrical sensors 834 can be on an exterior side of the detachable ear extensions 832 so as to lie adjacent to the wearer's skin and record data.

The eyeglass device 800 can also include a detachable band 836 with imbedded electrical sensors 838. These embedded electrical sensors 838 can be placed anywhere on the detachable band 836. The detachable band can secure the eyeglass device 800 to the wearer's head and lie flush against the wearer's skin so that the electrical sensors 838 can gather sensor data.

For the purposes of illustration, FIG. 8 has provided locations 802-826 for where specific sensors can be located. Additional or alternative sensor locations can be provided for anywhere on the eyeglass device 800 without limitation. Furthermore, where a specific type of sensor is provided for at a sensor location 802-826, any other type of sensor can be placed there as well. Additionally, there can be locations on the eyeglass device 800 for a battery, a WiFi connector element, a Bluetooth connector element, a USB port, and/or any other form of interfaces or modes of wired or wireless communication. Additional detachable components may be added to the eyeglass device 800 to provide additional sensors or to be used for comfort.

In some instances of the present disclosure, the eyeglass device 800 can include a GPS sensor. Data collected from the GPS sensor can facilitate finding a person in the event a seizure is detected. Data collected from the GPS sensor can also aid in seizure detection. Seizures are often associated with confusion, which may, for example, cause a person to wander around, or geographically go “off track” from their usual daily routine. Information from GPS can be used by the algorithm to determine whether the person is following their normal behavior.

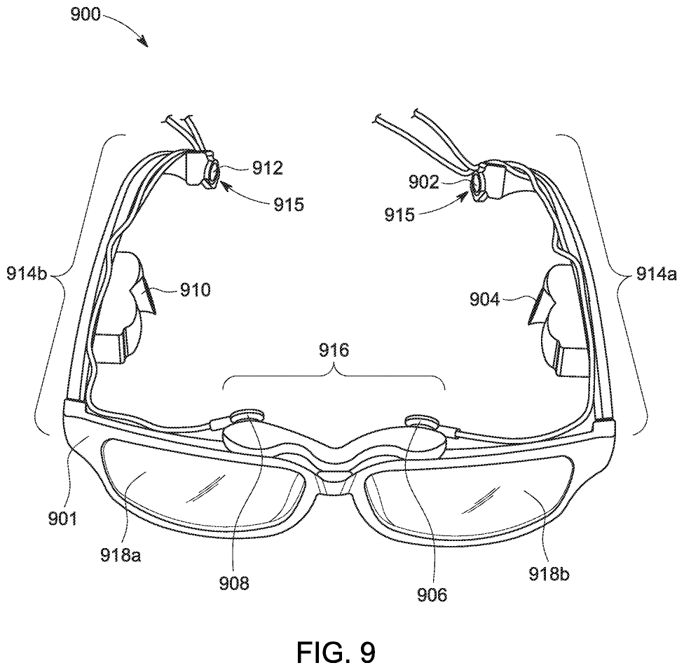

FIG. 9 shows an eyeglass device 900, which is another exemplary embodiment of the disclosed wearable device. Eyeglass device 900 includes a frame 901, a first electrode 902, a second electrode 904, a third electrode 906, a fourth electrode 908, a fifth electrode 910, a sixth electrode 912, a first temple portion 914a, a second temple portion 914b, a bridge portion of the frame 916, and lenses 918a and 918b.

The frame 901 is an eyeglass frame, which includes electrodes 902, 904, 906, 908, 910, and 912. In some examples, the electrodes 902, 904, 906, 908, 910, and 912 are permanently affixed to the frame 901; in other examples, some or all of the electrodes 902, 904, 906, 908, 910, and 912 are removeable and/or repositionable. The frame 901 further houses lenses 918a and 918b. In some examples, lenses 918a and 918b are corrective lenses. In some examples, lenses 918a and 918b have a diopter step of 0.

The first electrode 902 and the sixth electrode 912 are positioned on end portions 915 of temple portions 914a and 914b (respectively) of the frame 901; the second electrode 904 and the fifth electrode 910 are positioned on middle portions of temple portions 914a and 914b (respectively) of the frame 901; and the third electrode 906 and the fourth electrode 908 are positioned on a bridge portion of the frame 918. In some examples, end portions 915 correspond to a visual cortex of the wearer.

One exemplary electrode configuration is shown in FIG. 9. The contemplated electrode positions are selected to provide EEG data corresponding to relevant portions of the wearer's brain to provide seizure data. Although particular positions are shown in FIG. 9, the electrodes 902, 904, 906, 908, 910, and 912 can be placed in approximately similar positions, as would be readily contemplated by one skilled in the art. For example, each electrode 902, 904, 906, 908, 910, and 912 can be moved to the right or the left along the frame up to 1 centimeter.

The electrodes 902, 904, 906, 908, 910, and 912 can be communicatively coupled to an EEG monitoring machine (not shown). In some examples, the electrodes 902, 904, 906, 908, 910, and 912 are wired directly to the EEG monitoring machine; in other examples, the electrodes 902, 904, 906, 908, 910, and 912 are configured to wirelessly communicate with the EEG monitoring machine. For example, the first electrode couples to a P3 input; the second electrode couples to a C3 input; the third electrode couples to an Fp1 input; the fourth electrode couples to an Fp2 input; the fifth electrode couples to a C4 input; and the sixth electrode couples to an A2 input. Therefore, by the coupling between the electrodes 902, 904, 906, 908, 910, and 912 and the EEG machine, system 900 is configured to monitor EEG data of the brain of a subject. In some examples, the EEG machine includes any computing device configured to receive and process EEG data, including, for example, a smartphone.

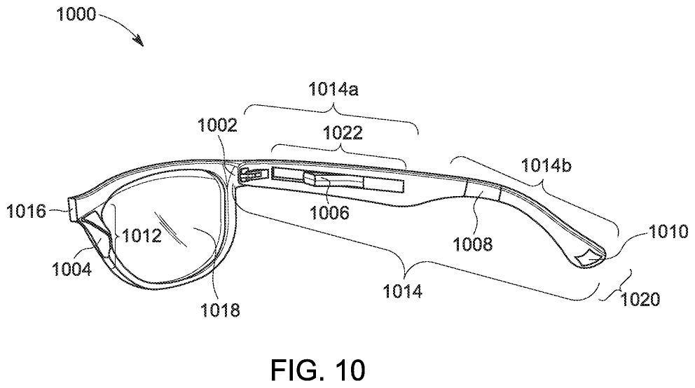

FIG. 10 shows a cut-away view of an eyeglass device 1000, which is another exemplary embodiment of the disclosed wearable device. Eyeglass device 1000 may include a frame 1002, a first electrode 1004, a second electrode 1006, a third electrode 1008, a fourth electrode 1010, a nosepiece 1012, a temple portion 1014, a bridge 1016, a lens 1018, an end portion 1020, and a track 1022.

FIG. 10 shows half of an exemplary eyeglass frame 1002 with four electrodes 1004, 1006, 1008, and 1010; the present disclosure contemplates that an opposing half of the exemplary eyeglass frame (not shown) includes four additional corresponding electrodes to yield an eyeglass device with eight total electrodes. Additional or fewer electrodes are further contemplated, as would be readily understood by one skilled in the art.

Electrode 1004 is positioned on a nosepiece 1012 of the frame 1002. In some examples, electrode 1004 has a corresponding shape to a shape of the nosepiece 1012. Therefore, electrode 1004 is configured to lie flush with the nose of the user and receive biometric data corresponding to the user. In some examples, electrode 1004 is positioned along a bridge portion 1016 of the frame 1002.

Electrode 1006 is positioned along a first portion 1014a of a temple portion 1014 of the frame 1002. Electrode 1006 protrudes from the frame 1002 such that electrode 1006 contacts the user's head. In some examples, electrode 1006 is repositionable along a track 1022. For example, although electrode 1006 is positioned in the center of track 1022, a user can slidably move electrode 1006 to any position along the track 1022. Therefore, adjustable electrode 1006 is configured to move positions depending on a user's head size and shape.

Electrode 1008 is positioned along a second portion 1014b of a temple portion 1014 of the frame 1002, and electrode 1010 is positioned along an end portion 1020 of the temple portion 1014 of the frame 1002. In some examples, the end portion 1020 corresponds to a visual cortex of the wearer when the device 1000 is worn by a user. In some examples, as shown in FIG. 10, both electrodes 1008 and 1010 are embedded into the frame 1002 so that they are flush with an external surface of the frame 1002. In other examples, one or both of electrodes 1008 and 1010 protrude from the frame 1002 to directly contact with the user's head. Although particular positions of electrodes 1008 and 1010 are shown in FIG. 10, electrodes 1008 and 1010 can be moved right or left along the temple portion 1014 up to a centimeter. In some examples (not shown), electrodes 1008 and 1010 slidably positionable on tracks (e.g., corresponding to track 1022).

In some examples, the frame 1002 is made of a pliable and resilient material, such that the temple portions 1014 of the frame 1002 can be bent by a user into a new shape.; the frame 1002 is then resilient enough to retain the new shape. This can help position the electrodes to contact the user's skin or apply more pressure to get a better signal to noise ratio. In some examples, the frame 1002 comprises a soft plastic shell and an interior wire frame, where the wire is reconfigurable into a new shape when sufficient force is applied. In some examples, the interior wire frame is made of a metal material, or other wire which provides communicative coupling between the electrodes 1004, 1006, 1008, and 1010.

All devices of FIGS. 3 and 8-10 be intermingled and/or combined. For example, the present disclosure contemplates that an exemplary device includes one or more, in any combination, of the features of any of FIGS. 3 and 8-10.

In some examples, an exemplary device comprises a frame, a plurality of electrodes, and electronics enclosure communicatively coupled to the electrodes. The electronics enclosure includes a processor, a memory module, a communication element. In some examples, the communication element is wired or wireless. The electronics enclosure is communicatively coupled to the electrodes and other electronic components either through a thin wire or through wireless communication via the communication element.

### Electrode Structure

In some examples, any of the electrodes discussed with respect to FIGS. 3, and 8-10 can have various features discussed herein. In some examples, all electrodes on an exemplary device (e.g., devices 300, 800, 900, or 1000) are similar; in other examples, the electrodes have different, or slightly varying, combinations of the features discussed further herein.

In some examples, one or more of the disclosed electrodes on an exemplary device are detachable from the frame and repositionable in another position on the frame. For example, a first subset of the plurality of electrodes are detachable from the frame and repositionable in another position on the frame, and a second subset of the plurality of electrodes are permanently configured in a fixed position in the frame.

In some examples, one or more of the disclosed electrodes on an exemplary device are embedded within a frame and flush with an external surface of the frame. In some examples, one or more of the disclosed electrodes on an exemplary device protrude from an external surface of the frame so as to contact a user's head.

In some examples, one or more of the disclosed electrodes on an exemplary device are dry electrodes, foam electrodes, and/or made from conductive silicon or a conductive metal.

In some examples, one or more of the disclosed electrodes on an exemplary device are a polymer shape, including, for example, a circle shape, a square shape, a rectangular, and an ovoid shape.

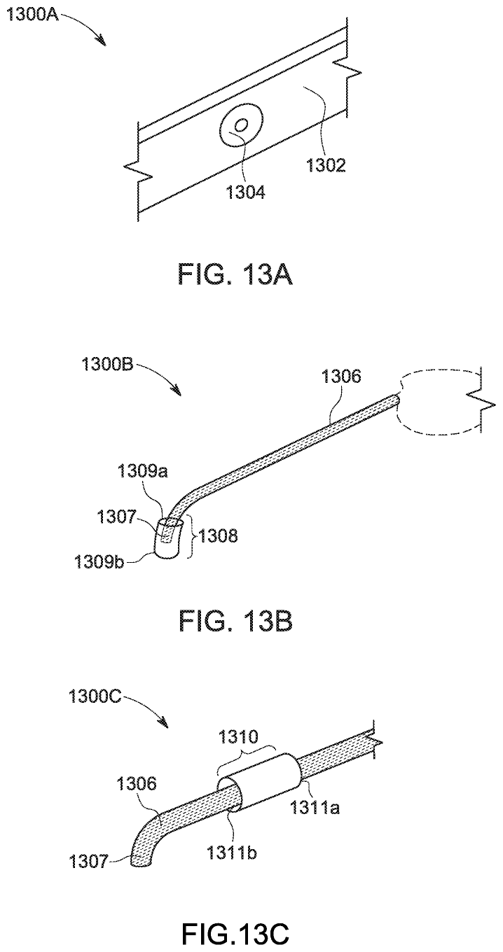

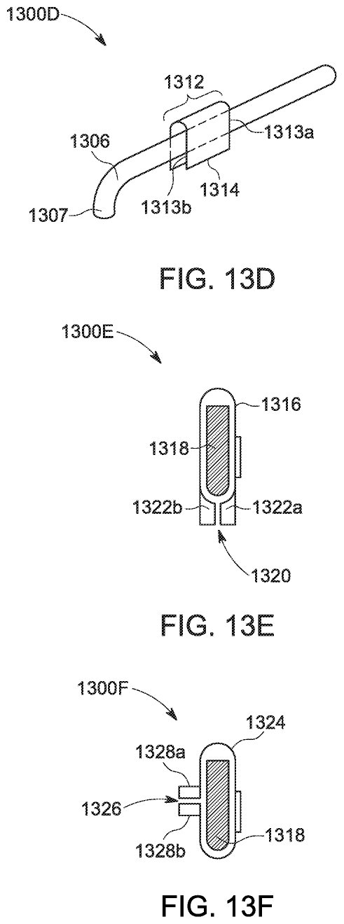

FIGS. 13A-13F show additional details of exemplary electrodes, according to various embodiments of the present disclosure. Any of the electrodes discussed with respect to FIGS. 13A-13F can be used in any of the devices discussed herein. In some examples, more than one of any of the electrodes shown in FIGS. 13A-13F are used in an exemplary device. In some examples, any combination of the electrodes shown in FIGS. 13A-13F are used in an exemplary device.

FIG. 13A shows an exemplary electrode configuration 1300A, which includes a frame portion 1302 and an electrode 1304. The frame portion 1302 is, for example, any segment of an eyeglass frame, including, any segment on temple portions of an eyeglass frame. The electrode 1304 is a circular shape, configured to snap into a receiving portion of frame 1302. Therefore, electrode 1304 is removable and replaceable by a user.

FIG. 13B shows an exemplary electrode configuration 1300B, which includes a temple portion of a frame 1306 and an electrode 1308. The electrode 1308 is configured as a hollow element with an open end 1309a and a closed end 1309b. The open end 1309a therefore receives an end portion 1307 of a temple portion of the frame 1306, and is configured to slide on until the end portion 1307 of the temple portion of the frame 1306 abuts the closed end 1309b of the electrode 1308. Therefore, electrode 1308 is removable and replaceable by a user.

FIG. 13C shows an exemplary electrode configuration 1300C, which includes a temple portion of a frame 1306 and an electrode 1310. The electrode 1310 includes two open side portions 1311a and 1311b; the open side portions 1311a and 1311b allow the electrode 1310 to slide along the temple portion of a frame 1306. In some examples, electrode 1310 is removed by sliding off an end portion 1307 of the temple portion of the frame 1306. For example, electrode 1310 is a tubular shape. Therefore, electrode 1310 is repositionable and removeable.

FIG. 13D shows an exemplary electrode configuration 1300D, which includes a temple portion of a frame 1306 and an electrode 1312. The electrode 1312 includes two open side portions 1313a and 1313b; the open side portions 1313a and 1313b allow the electrode 1312 to slide along the temple portion of a frame 1306. In some examples, electrode 1312 is removed by sliding off an end portion 1307 of the temple portion of the frame 1306. For example, electrode 1312 is a c-shape, a hook shape, and/or a u-shape. Electrode 1312 has an open bottom portion 1314, which allows electrode 1312 to snap onto and snap off of the temple portion of the frame 1306. Therefore, electrode 1312 is repositionable and removeable.

FIG. 13E shows a cut-away view of exemplary removable electrode configuration 1300E, which includes an electrode 1316 and a frame portion 1318. The frame portion 1318 is, for example, any segment of an eyeglass frame, including, any segment on temple portions of an eyeglass frame. The electrode 1318 includes an opening 1320 and two stopper portions 1322a and 1322b. The electrode 1318 is configured to snap onto frame portion 1318 by sliding the frame portion 1318 through the opening 1320. The two stopper portions 1322a and 1322b secure the electrode 1316 on the frame portion 1318. Electrode 1316 is further configured to slide horizontally along frame portion 1318. Therefore, electrode 1316 is repositionable and removeable.

FIG. 13F shows a cut-away view of exemplary removable electrode configuration 1300F, which includes an electrode 1324 and a frame portion 1318. The frame portion 1318 is, for example, any segment of an eyeglass frame, including, any segment on temple portions of an eyeglass frame. The electrode 1324 includes an opening 1326 and two stopper portions 1328a and 1328b. The electrode 1324 is configured to snap onto frame portion 1318 by sliding the frame portion 1318 through the opening 1326. The two stopper portions 1328a and 1328b secure the electrode 1324 on the frame portion 1318. Electrode 1324 is further configured to slide horizontally along frame portion 1324. Therefore, electrode 1316 is repositionable and removeable.

Although various electrode shapes, configurations, and attachment means are shown in FIGS. 13A-13F, the present disclosure contemplates various alterations of these features, as would readily be contemplated by one skilled in the art. In some examples, the electrodes are adhered to the frame via an adhesive, including, for example, double-sided tape or glue. In some examples, the adhesive is permanent or temporary, to allow user removal of the electrode.

The present disclosure further contemplates that the electrodes transmit the electrode data to any of the electronic elements discussed herein, including an electronic enclosure, a processor, a memory module, a communication element, and any combination thereof. In some examples, the electrodes transmit electrode data (1) wirelessly, (2) through an external wire directly coupling the electrode to one or more of the electronic elements, (3) through an external wire directly coupling the electrode to one or more of the electronic elements, (4) through an internal wire embedded in the frame, which directly couples the electrode to one or more of the electronic elements, (5) through an internal wire embedded in the frame, which indirectly couples the electrode to one or more of the electronic elements, and (6) any combination thereof. In some examples, indirect coupling of an internal or external wire occurs through a portion of the wire contacting a conductive material, and the conductive material contacting the electrode. In some examples, the conductive material includes a conductive adhesive.

### Biological Data Pattern Recognition

FIG. 4 is a flow chart illustrating a methodology 400 for monitoring brain function and health, according to an exemplary embodiment of the present disclosure. The methodology begins at step 410 when a brain health system receives EEG data from a plurality of sensors on a wearable head apparatus. The EEG data can comprise electrical signals representing brain activity of a user wearing the wearable head apparatus. In other examples, the data received can be other biological health data and not necessarily EEG data, or non-biological data altogether. In other examples, the method can receive EEG data, additional biological data, and non-biological data, or any combination of such data. For example, step 410 can include receiving video data from a camera associated with the brain health system, movement data from a motion sensor (e.g., an accelerometer) associated with the brain health system, and/or audio data from a microphone associated with the brain health system.

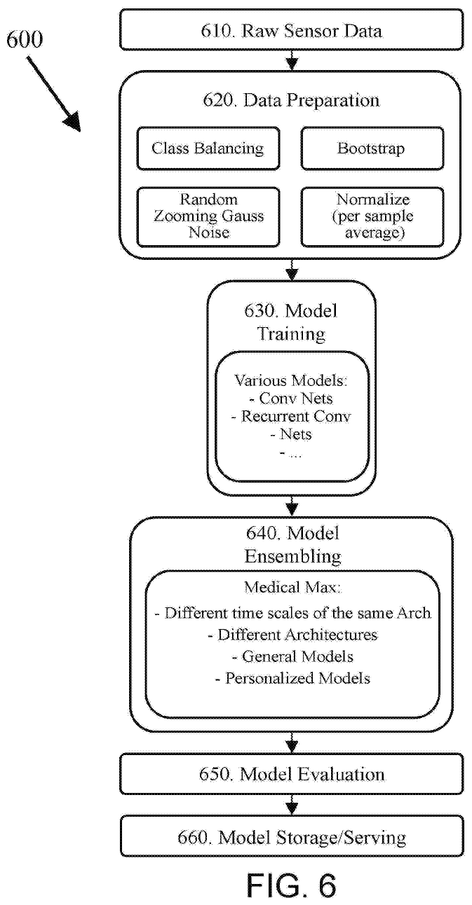

After receiving the EEG data, the method 400 proceeds in step 420 by processing the received data using a machine learning model. An example of this processing can be discussed further with respect to FIG. 6. Referring back to FIG. 4, the method 400 functions to analyze the data and identify a time window representing a seizure. The analysis can be performed through a machine learning model, and a convolutional neural network, in particular.

In some examples, each type of data has a separate machine-learning model, including, for example, a first machine learning model for processing EEG data, a second machine learning model for processing audio data, a third machine learning model for processing visual data, and any other machine learning model as needed. In some examples, a machine learning model receives more than one type of input data, including for example, audio data and EEG data; visual data and EEG data; visual data, audio data, and EEG data; or any combination of data types as discussed herein.

The method 400 can identify a time window representing a seizure based on a pattern of the EEG data which is similar to a confirmed EEG seizure pattern. The method 400 can have a threshold similarity metric to identify a proposed time window should be identified as representing a seizure. The identified seizure can be convulsive or non-convulsive.

In some examples, step 420 provides for comparing time windows identified for different types of data. For example, step 420 provides for determining (1) whether a time window identified based on visual data corresponds to a time window identified based on EEG data, (2) whether a time window identified based on audio data corresponds to a time window identified based on EEG data, (3) whether a time window identified based on movement data corresponds to a time window identified based on EEG data, (4) whether a time window identified based on audio data corresponds to a time window identified based on visual data, (5) whether a time window identified based on movement data corresponds to a time window identified based on visual data, (6) whether a time window identified based on audio data corresponds to both a time window identified based on EEG data and a time window identified based on visual data, (7) whether a time window identified based on visual data corresponds to both a time window identified based on EEG data and a time window identified based on audio data, (8) whether a time window identified based on EEG data corresponds to both a time window identified based on audio data and a time window identified based on visual data, and/or (9) whether a time window identified based on EEG data corresponds to a time window identified based on audio data, a time window based on movement data, and a time window identified based on visual data.

In some examples, step 420 provides for outputting a notification based on the comparison of time windows. For example, the notification identifies for a user, a caretaker, or a health professional whether or not the identified windows correspond with each other.

The method can then proceed to step 430. The method will tag the identified time window as seizure data. The associated seizure data can include an intensity of the seizure, a biological response of the user of the wearable head apparatus, a time of the seizure, and all biological data recorded by sensors before, during, and after the seizure event. The tagged time window can be used to further train the machine learning model as labeled data identifying what a seizure can look like.

In step 440, the method 400 completes by outputting a representation of the time window as seizure data. This representation can be sent to a mobile device of the user, to a remote server, or to a caretaker of the user. The representation can include data about the seizure such as a time the seizure occurred, a severity of the seizure, biological response data of the user before, during and after the seizure, and any other similar data. In some instances, the representation can include a warning that a seizure is about to begin. The warning can be based on processed EEG data from the sensors.

Alternatively, or in addition, the representation can be sent to emergency professional. For example, the method 400 can determine whether the seizure event is sufficiently severe such that the user of the wearable head apparatus needs immediate medical assistance. The determination can be made based on the EEG data sent by the sensors. The representation can include informational data about the user to assist the emergency personnel with providing appropriate treatment for the user. This informational data can include information about the user such as location, age, seizure state, and seizure history.

In some examples of methodology 400, the data is processed to identify any abnormal brain activity, and not just seizure data. For example, the data is processed to identify EEG markers associated with Alzheimer's, Parkinson, and/or Autism. Therefore, some embodiments of the present disclosure provide for identifying, tagging, and outputting representation of a time window corresponding to abnormal brain activity or an event associated with Alzheimer's, Parkinson, and/or Autism.

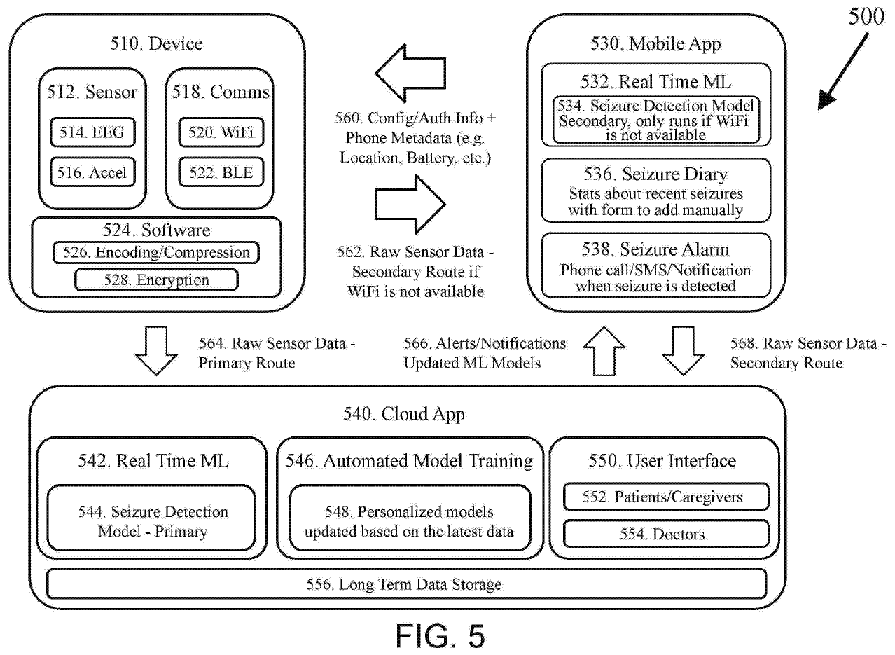

FIG. 5 is a diagrammatic view of a health monitoring system and data exchange 500, according to an exemplary embodiment of the present disclosure. FIG. 5 provides a view of the brain health monitoring system 500 actively reviewing, analyzing, and transmitting data to provide seizure detection and review for a user.

The device 510 is the element worn by the user which serves to receive biological data from the user. The device 510 can have sensors 512 which measure EEG data 514 of the user and gravitational acceleration 516 of the device 510. The data captured by the sensors can be referred to as raw sensor data. The device 510 can also have software 524 which encodes and compresses 526 the raw sensor data. The compression 526 allows large amounts of data to be easily transferred to another element of the system. The device 510 can also encrypt 528 the data for protection of the raw sensor data during transfer. The encryption 528 of the data protects the user's private health information. The device 510 has communication elements 518 such as a Wi-Fi communication element 520 and/or a Bluetooth communication element 522. The software 524 can send the raw sensor data to another element of the system via the Wi-Fi communication element 520 or the Bluetooth communication element 522.

A primary route 564 of the raw sensor data is transmittal via the Wi-Fi communication element 520 to an application in the cloud 540. The cloud application 540 provides real time machine learning 542 to predict or detect seizures, and monitor brain health in general. The real time machine learning 542 receives the raw sensor data runs it through a seizure detection model 544. The seizure detection model 544 determines whether the raw sensor data is similar enough to seizure data to identify a seizure event during a time window of the raw sensor data. When a seizure is detected, the cloud application 540 can send alerts and notifications 566 to a mobile application. The real time machine learning 544 is discussed further with respect to FIG. 6.

Referring back to FIG. 5, the cloud application 540 can also provide for automated model training 546. The automated model training 546 can personalize the machine learning model used to predict and detect seizures based on the raw sensor data from the device. This creates a personalized adaptive machine learning model 548 based on biological data from the user. This can also ensure a higher accuracy of the machine learning model because it is based on personalized data from the user instead of generic data from an accumulation of other individuals. Therefore, automated model training 546 allows for better prediction and detection of when the user is actually experiencing a seizure instead of just identification of when other, unrelated individual might experience a seizure based on the raw sensor data. When the machine learning model 542 is updated based on biological data from the user, the updated model 566 can be sent to the mobile application.

The cloud application 540 can also provide for a user interface 550. This allows patients/users, caregivers, and doctors to access the raw sensor data, the model training, and the detected seizure data. Patients/users and caregivers 552 can have separate user interfaces from doctors 554. For example, the interface can provide alerts or notifications 566 sent to a mobile application 530 when a seizure event is detected. In some examples, the user interface 550 can give the patient, caregiver, doctor, and/or any health care provider the ability to confirm or deny that a seizure event took place during a seizure event detected by the real-time machine learning 542. In other examples, the user interface 550 can allow the patient, caregiver, doctor, and/or any caregiver to identify that a seizure did occur during a certain timeframe, when the cloud application 540 did not detect a seizure during that timeframe. In all instances, the user interface 550 can send the corrections to the automated model training 546 to then update the machine learning model.

The user interface can have a separate doctor interface. The doctor interface can, for example, provide the ability to doctor to over-ride any actions made by the user. For example, the user may accidental push the button marking a seizure that did not happen. A doctor can confirm this and remove the data from machine learning input. Additionally, patients can give permissions to others to review/view their data, including EEG, video, GPS location, accelerometer, heart rate, etc. While patients access their “device” page, doctors have access to all the data to which they have been given permission. There are several types of permissions too: HISTORIC_<data_type> (data >6 mo), REAL_TIME_<data_type> (data <6 mo), ONLINE_STATUS (whether the patient is using the device), RECEIVE_ALARM (whether they can be notified of abnormal events, such as seizures, STATISTICS (whether they can see summary statistics including seizure counts and times). The user can choose to give another user (generally a doctor) permission by adding their email and checking the permissions they want to allow.

The cloud application 540 can also provide for long term data storage 556. The long term data storage 556 can hold all raw sensor data output from the device 510. The long term data storage 556 can hold corrections of predicted and detected seizures from the user interface 550. Long-term data can include: (1) EEG segments of events of interest; (2) seizure counts, (3) seizure durations, (4) seizure intensity, (5) seizure spread or location (i.e., where seizure started, and where it went in the brain); (6) min/max heart rate, (7) min/max oxygenation, (8) EDA changes. Any other snapshot of any of the collected data, including video, still images, sounds, location, and accelerometer data can also be stored. In some instances, the long-term data can be stored on a mobile device or the wearable head apparatus.

A secondary route 568 for the raw sensor data detected from the device 510 can be to a mobile application 530. The raw sensor data can be sent via Bluetooth 522. For example, the raw sensor data can be transmitted via the secondary route 568 if Wi-Fi 520 is not available for the device 510. The mobile application 530 can have a real time machine learning model 532 on the mobile application 530 to detect whether a seizure occurred based on the raw sensor data. Detection can occur through a seizure detection model 534 in the mobile application 530. However, the mobile application 530 can prefer to send the raw sensor data to the cloud application 556 via Wi-Fi instead of running the machine learning model 532 on the mobile application 530. If the cloud application 540 receives the raw sensor data from the mobile application 530, the cloud application 540 can run its real time machine learning 542 to detect whether a seizure occurred. If Wi-Fi is not available, the mobile application 530 can run the real-time machine learning 532 on the mobile device.

The mobile application 530 can have a seizure diary 536 available for the user to interact with. The seizure diary can include statistics about recent seizures. For example, the statistics can include seizure information such as time, location, severity, length, and other data. Beyond count and duration data, a timeline can break down seizures per month, week, and day, including time of day. This facilitates the discovery of behavioral patterns that induce seizures. Additionally, the data can include: hours that the wearable head apparatus is connected, a number of seizures alerted, a number of seizures without device, and a number of seizures mislabeled by patient. Latency of detection per seizure, length of time before seizure for prediction. The seizure diary 536 can include a location for the user to indicate that a seizure did not actually occur during a detected time window. Additionally, the seizure data can include a form for the user to manually add that a seizure did occur during a time window where a seizure was not detected, or to remove events that were incorrectly detected as seizures when a seizure did not actually occur.

The mobile application 530 can also provide a seizure alarm system 538. For example, if a seizure is detected, the mobile application 530 can provide for the mobile device of the user to ring, for the user to receive a text message, or for the user to receive any other sort of notification on a mobile device of the user. The mobile application can also be configured to send alerts to other entities such as emergency services, caregivers, healthcare providers, when a seizure is detected or predicted. In some examples of the present disclosure, the device 510 can be configured to send notifications independently to the user when the device 510 is connected to a network. Therefore, notifications do not need to pass through the mobile application.

The mobile application 530 can send configuration/authorization information 560 and mobile phone metadata 560 to the device. For authorization, each user (patient/doctor/caregiver) can have an identity that is unique and kept safe on secure servers. When a user successfully logs in to a web/mobile app the server provides the user with a cryptographically verifiable token that attests to that identity of the user and includes some of the user's permissions, including whether they have access to the device (each device can have a unique serial number). The device can verify the user's identification by matching the cryptographic signature with the token and then checking if the token includes permission to configure the device. After this authentication, the device can allow the exchange of configuration between the mobile/web app and the device.

In order to configure the device, the device can be associated with a user, types of data can be selected to stream to server, and a battery profile (normal (normal data rates, high (high data rates), battery saver (low data rates) can be selected. For example, the mobile phone metadata 560 can include a location of the mobile phone, information on a battery status of the mobile phone. For example, the phone and device can know about each other's model numbers or version, in order to facilitate some degree of automatic configuration for communication. Additionally, accelerometer data from the phone, along with location, can be features in the machine learning algorithms.

### Machine Learning

In some examples, the statistical analysis utilized to implement various features disclosed in the system 100 of FIG. 1 can be a machine learning or artificial intelligence algorithm. Machine learning algorithms may take a variety of forms. For instance, the system 100 may utilize more machine learning algorithms including (1) artificial neural networks (ANN), (2) deep neural networks (DNN), (3) convolutional neural networks (CNN), or (4) recurrent convolutional neural networks (RCNN).

Artificial neural networks (“ANN”) are computational models inspired by a biological central nervous system (or brain). They map inputs to outputs through a network of nodes. The nodes do not necessarily represent any actual variable. Accordingly, ANN may have a hidden layer of nodes that are not represented by a known variable to an observer. ANNs are capable of pattern recognition and have been used for the medical and diagnostics fields. Their computing methods make it easier to understand a complex and unclear process that might go on during diagnosis of an illness based on input data a variety of input data including symptoms.

DNN is a relatively new type of machine learning algorithm that is capable of modeling very complex relationships that have a lot of variation within the relationships. For example, DNN has developed recently to tackle the problems of speech recognition. In the IT (information technology) industry fields, various architectures of DNN have been proposed to tackle the problems associated with algorithms such as ANN by many researchers during the last few decades. These types of DNN are CNN (Convolutional Neural Network), RBM (Restricted Boltzmann Machine), LSTM (Long Short Term Memory) etc. They are all based on the theory of ANN. They demonstrate a better performance by overcoming the back-propagation error diminishing problem associated with ANN.

EEG data in general, and large-scale EEG data in particular, is very dense and noisy. Traditional machine learning techniques are very computationally demanding and cannot efficiently analyze the raw data online in a manner that is timely enough for an alarm to be of use to patients and caregiver. Simpler approaches, like basic band pass filters are too prone to false positives to be useful. Other machine learning algorithms relied on feature engineering, which leverages domain knowledge to transform and summarize the data to reduce its size and dimensions and feed it to simpler models. None of these traditional approaches are successful, due to their lack of ability to correctly distinguish between seizure and non-seizure patterns online.

By contrast, the present disclosure relies on CNNs. CNNs are a type of neural network that uses mathematics similar to mathematics used to render computer graphics, on graphics processing units (GPUs). It is extremely computationally and economically efficient to run these networks even on very dense, noisy data, such as EEG. In the present disclosure, 1D convolutions summarize the distribution of the data, and then feed higher dimensional convolutions that capture the relationship between the patterns between series. In practice this allows the processing of EEG data even in its raw form, where it is the extremely dense and noisy. This raw form also is the richest in information as no information has yet been lost in transformations. This allowed us to have very high scores for our models (0.988 Area under the receiver characteristic curve for detection) on very large numbers of patients (>200). Not only is the model computationally efficient to process the most information rich version of the data, but the model is also rich enough to capture patterns from many individuals allowing very high scores despite the large population.

On top of regular CNN, RCNN, or recurrent CNN introduce a factor of time. In these cases, the CNN is effectively unfolded in the time dimension allowing not only the analysis of a static EEG patterns, but the evolution of this pattern over time and over different time scales. While detection performs pretty well with CNN, prediction is a much harder problem, concerned about much more nuanced features. RCNN makes a difference, and can increase the accuracy beyond CNNs by ˜5-10% or more. In a lot of real world cases, this is the difference between an unusable system that emits many false positives and a usable one.

### Machine Learning—Training Data

Machine learning algorithms require training data to identify the features of interest that they are designed to detect. For instance, various methods may be utilized to form the machine learning models including applying randomly assigned initial weights for the network and applying gradient descent using back propagation for deep learning algorithms. In other examples, a neural network with one or two hidden layers can be used without training using this technique.

In some examples, the machine learning algorithms will be trained using labeled data, or data that represents certain features or characteristics, including EEG data representing a seizure, accelerometer data indicating a convulsion, and other features. In some examples, the training data will be pre-filtered or pre-analyzed to determine certain features, including various high level filters or starting points that include motion sensing or baseline EEG data. In other examples, the data will only be labeled with the outcome and the various relevant data may be input to train the machine learning algorithm.

FIG. 6 is a diagrammatic view of a process 600 for training and selecting a machine learning model serving to detect seizures, according to an exemplary embodiment of the present disclosure. In step 610, the process 600 can receive raw sensor data from a wearable head apparatus which measures biological data.

In step 620, the raw sensor data can then go through data preparation. The data preparation step 620 provides pre-processing of the data to better train the model. Providing cleaned, normalized data enables model training occurs more smoothly because clean data helps the machine learning model to more easily identify a seizure event from a non-seizure event. The process 600 can apply a variety of methods to clean the data, including class balancing the data, bootstrapping the data, normalizing the data per sample average, and accounting for random zooming Gauss noise.

After the data is cleaned in step 620, the process 600 can then proceed to step 630 where the model is trained. In this step, the process 600 trains various models with the data prepared from step 620. For example, step 630 can train machine learning algorithms including (1) artificial neural networks (ANN), (2) deep neural networks (DNN), (3) convolutional neural networks (CNN), or (4) recurrent convolutional neural networks (RCNN).

The process 600 can then proceed to step 640 to complete model ensembling. Model ensembling determines how machine learning models are selected and applied to specific users. For example, model ensembling can assign a general model which is a machine learning algorithm generalized for all users. In other example, model ensembling can assign a personalized model which includes specific variables just for one user. This case is more likely where the user has enough data to train the model. In another example, step 640 can use different time scales which can select larger or smaller windows of EEG data to train the machine learning model. Additional examples of model ensembling include using the medical max and using different architectures.

The process can then proceed to step 650 where model evaluation is completed. In this step, the process 600 can evaluate the accuracy of the chosen machine learning model.

The process 600 then proceeds to step 660 where the model completes storage and serving. Model storage occurs when the chosen machine learning model is stored until it is ready to be used to process sensor data to identify whether there is a seizure event. Model serving is when the device is retrieved by a processing device. Sensor data can be fed through the machine learning model to identify whether there was a seizure event during the time window of the sensor data.

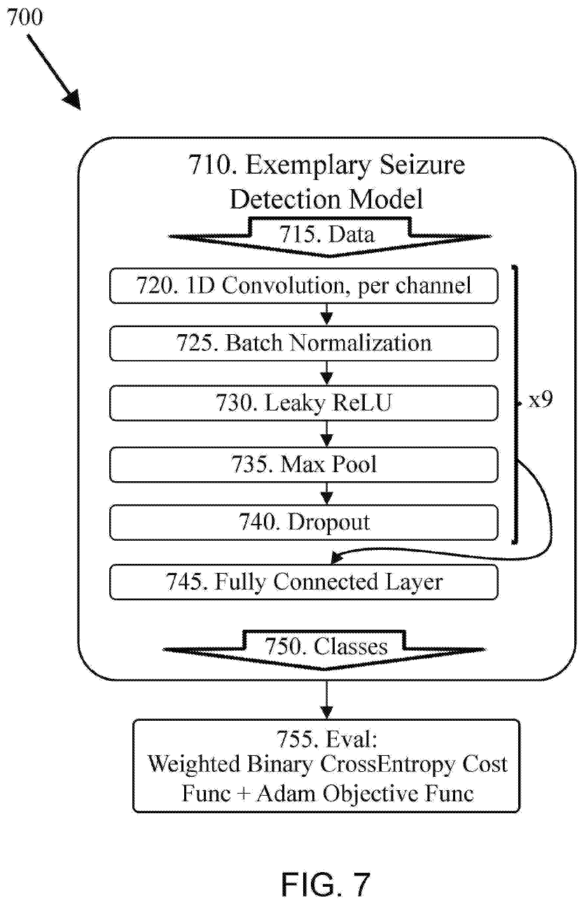

FIG. 7 is a diagrammatic view of an exemplary model 700 for predicting or detecting a seizure once a machine learning model has been trained and selected according to FIG. 6. A seizure detection model 710, according to an embodiment of the present disclosure, can start at step 715 by running sensor data through a machine learning model. In steps 720 through 750, the machine learning model runs a variety of processes on the data. The processes can start at step 720 with running a one dimensional convolution on the data. One dimensional convolution can examine the biological data, such as the EEG signals, to make assumptions about relationships with the data and identify similar patterns in different sets of data points. For example, the similar patterns that the one dimensional convolution identifies in the data can be seizure events. One dimensional convolution can work well with fixed lengths of data. For example, the present disclosure can provide for fixed time windows of the biological data.

The seizure detection model 710 can proceed to batch normalization in step 725. Batch normalization can reduce covariance shift and reduce the amount of data that needs to be dropped out in later steps. Batch normalization can function by subtracting the batch mean and dividing by the batch standard deviation. This increases the stability of the seizure detection model 710.

The seizure detection model 710 can then proceed to a leaky version of a rectified linear unit (“Leaky ReLU”). Rectifiers allow better training of deeper networks by working as an activation function. The Leaky ReLU can set biases to small positive values. This can minimize losses in the training data.

The seizure detection model 710 can then proceed to max pooling as a sample-based discretization process. Max pooling can apply a filter over the initial data and select the maximum value in that region. Max pooling reduces the amount of data that the model is learning from and can help reduce over-fitting of seizure events by looking at the data in a more abstract manner.

The seizure detection model 710 can then proceed to dropout in step 740. Dropout allows certain characteristics of the data to be omitted from the model 710. Omitting certain characteristics can help break up situations where some characteristics of the data are influencing how the model 710 reviews other data characteristics. Dropout allows the model 710 to have a number of trained models sharing the same parameters.

The seizure detection model 710 can repeat steps 720-740 a set number of times in order to best train the model 710. For example, there can be nine repetitions. The model can then proceed to fully connected layer 745 after the final repetition of steps 720-740. Fully connected layers can connect to all activations in previous layers. For example, a previous layer can be a previous step of the seizure detection model 710.

Next, at step 750, the seizure detection model 710 can reduce the biological data to class scores. Lastly, in step 760, the seizure detection model 755 evaluates the weighted binary cross entropy cost function and the Adam objective function. In step 750, the output of the fully connected layer (the last layer of the network) can be interpreted as the classes “Seizure/Non-Seizure”. Given a pre-trained model, incoming EEG online can be analyzed to determine whether a seizure, or the telltale signs of a future seizure, have been detected.

Next, at step 755, the seizure detection model 710 can train the data. Weighted binary cross entropy is a scoring function to measure the distance between a model's predictions and the ground truth. Weighting the model can be added to fight the imbalance of the classes. For example, there can be many more occurrences of non-seizure than seizure and the model can respond by giving the seizure events more importance than their distribution in the data would naturally confer. This is a novel technique in its application to EEG analysis. The Adam objective function is an exemplary strategy to pick the (multidimensional) direction and value by which to change the weights of the connections between neurons during the training of the network.

This feedback, closed-loop, or adaptive nature of the seizure detection model 710 allows refining of the machine-learning algorithm. The model can be refined or retrained based on updated input from the user (e.g., falsely detected seizure, or a missed seizure). This increases the accuracy and utility of the seizure detection model 710.

### Experimental Data

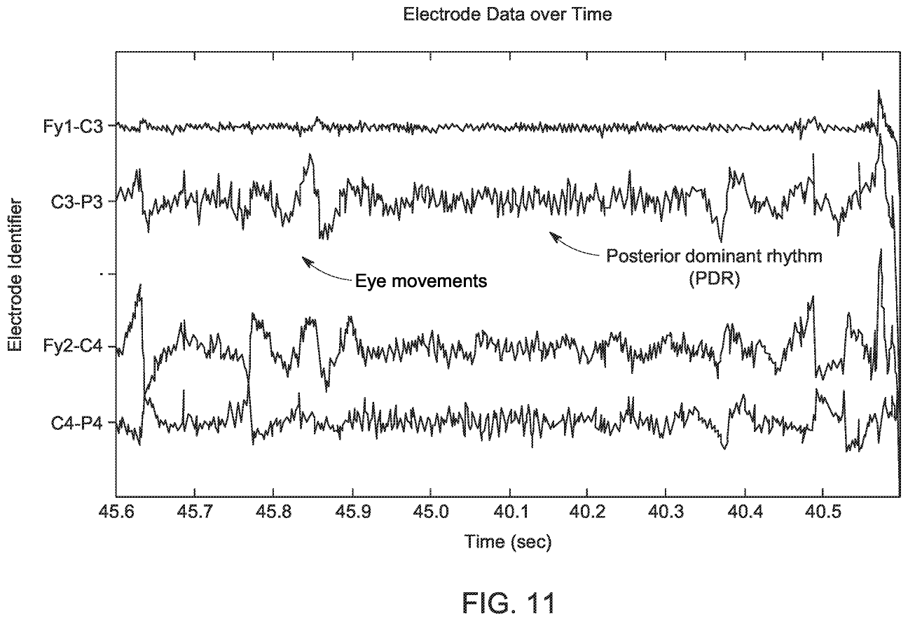

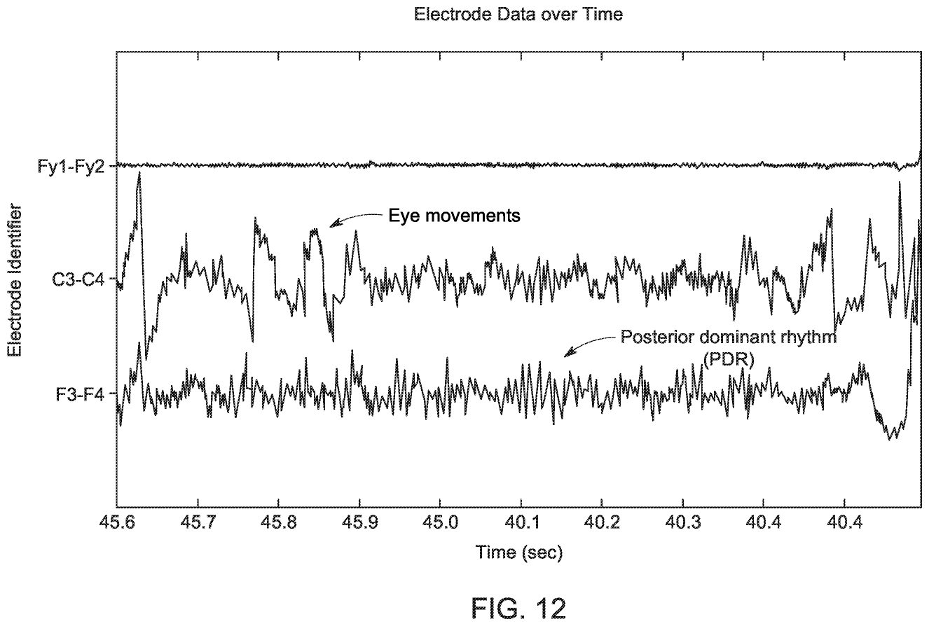

FIGS. 11-12 demonstrate the effectiveness of an exemplary device, according to the present disclosure, collecting brain activity which shows seizure events. The x-axis is time, and the y-axis is the amplitude of the signal. FIGS. 11-12 demonstrate the capability of the device to collect data from normal brain activity (e.g., the regions labeled posterior dominant movement), which is significantly more subtle than the activity related to epileptic seizures (e.g., the regions labeled eye movements).

### Conclusion

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A brain health system for monitoring brain function and health, comprising:
a wearable head apparatus;
a plurality of sensors;
a memory device containing machine readable medium comprising machine executable code having stored thereon instructions for performing a method of determining biological signals of a user of the wearable head apparatus;
a control system coupled to the memory device comprising one or more processors, the control system configured to execute the machine executable code to cause the one or more processors to:
receive electroencephalography (EEG) data output by at least one of the plurality of sensors, wherein the EEG data comprises electrical signals representing brain activity of the user; and
process the EEG data using a machine learning model to identify a time window of a subset of the EEG data representing a period of abnormal brain activity.

2. The brain health system according to claim 1, wherein the EEG data comprises a pattern, and the control system is further configured to execute the machine executable code to cause the one or more processors to identify a seizure of the user based on at least analysis of the pattern in the data output by the plurality of sensors.

3. (canceled)

4. The brain health system according to claim 1, wherein the biological signals are determined with respect to indications of a seizure in a brain of the user.

5. The brain health system according to claim 1, wherein the machine learning model is a convolutional neural network.

6. The brain health system according to claim 1, wherein the machine learning model is trained with labeled data that classifies whether a subject is experiencing a seizure during a subset of the labeled data.

7. The brain health system according to claim 1, wherein the control system is further configured to execute the machine executable code to cause the one or more processors to input data output from the plurality of sensors attached to the wearable head apparatus to determine the biological signals.

8. The brain health system according to claim 1, wherein the sensors are electrodes.

9. The brain health system according to claim 1, wherein the wearable head apparatus is an eyeglass device.

10. The brain health system according to claim 9, wherein the eyeglass device comprises a frame and a detachable band, wherein a subset or the entirety of the plurality of sensors can be located on the detachable band.

11. The brain health system according to claim 9, wherein the eyeglass device comprises a frame and a pair of detachable earpieces, wherein a subset or the entirety of the plurality of sensors can be located on the pair of detachable earpieces.

12. The brain health system according to claim 1, wherein the control system is further configured to:
tag the time window of the subset of the EEG data as seizure data; and
output a representation of the time window of the EEG data.

13. The brain health system according to claim 12, wherein the output representation comprises at least one of: an indication that the user is having a seizure and a prediction that the user will have a seizure.

14. (canceled)

15. The brain health system according to claim 1, wherein the wearable head apparatus further comprises a camera configured to record visual data of the user's face.

16. The brain health system according to claim 1, wherein the control system is further configured to:
receive visual data output from the camera; and
process the visual data using a machine learning model to identify a time window of a subset of the visual data representing a seizure.

17. The brain health system according to claim 16, wherein the control system is further configured to:
determine whether the identified time window of a subset of the visual data corresponds to the identified time window of a subset of the EEG data; and
output a notification, wherein the notification comprises the determination of whether the identified time window of a subset of the visual data corresponds to the identified time window of a subset of the EEG data.

18. (canceled)

19. The brain health system according to claim 1, wherein the control system is further configured to:
receive audio data output from the microphone; and
process the audio data using a machine learning model to identify a time window of a subset of the audio data representing a seizure.

20. The brain health system according to claim 16, wherein the control system is further configured to:
determine whether the identified time window of a subset of the audio data corresponds to the identified time window of a subset of the EEG data; and
output a notification, wherein the notification comprises the determination of whether the identified time window of a subset of the audio data corresponds to the identified time window of a subset of the EEG data.

21. The brain health system according to claim 1, wherein the wearable head apparatus further comprises an accelerometer configured to record movement data of the user.

22. The brain health system according to claim 1, wherein the control system is further configured to:
receive movement data output from the accelerometer; and
process the movement data using a machine learning model to identify a time window of a subset of the movement data representing a seizure.

23. The brain health system according to claim 16, wherein the control system is further configured to:
determine whether the identified time window of a subset of the movement data corresponds to the identified time window of a subset of the EEG data; and
output a notification, wherein the notification comprises the determination of whether the identified time window of a subset of the movement data corresponds to the identified time window of a subset of the EEG data.
